(19) United States
(12) Patent Application Publication
(10) Pub. No.: US 2024/0185420 A1
(43) Pub. Date: Jun. 6, 2024

(54) X-RAY BASED DENTAL CASE ASSESSMENT

(71) Applicant: Align Technology, Inc., San Jose, CA (US)

(72) Inventors: Christopher E. CRAMER, Durham, NC (US); Oscar Borrego HERNANDEZ, Morrisville, NC (US); Guotu LI, Apex, NC (US)

(21) Appl. No.: 18/527,206

(22) Filed: Dec. 1, 2023

Related U.S. Application Data
(60) Provisional application No. 63/429,545, filed on Dec. 1, 2022.

(51) Int. Cl. G06T 7/00; A61B 6/00; A61B 6/51; A61C 7/00; G06T 7/11; G16H 20/40

(52) U.S. Cl. CPC G06T 7/0014; A61B 6/51; A61B 6/5205; A61C 7/002; G06T 7/11; G16H 20/40; G06T 2207/10116; G06T 2207/20021; G06T 2207/20081; G06T 2207/20084; G06T 2207/20132; G06T 2207/30036

(57) ABSTRACT

Various apparatuses are disclosed (e.g., system, device, method, and the like) for assessing a dental x-ray image and determining, based on the dental x-ray, whether a patient is a candidate for a dental treatment. The apparatuses may use one or more trained neural networks to assess a patient's x-ray images and provide a recommendation for receiving a dental treatment. The neural networks may be trained based on a data training set of x-ray images that may and accompanying dental assessment data describing one or more dental characteristics.

### CLAIM OF PRIORITY

This patent application claims priority to U.S. Provisional Patent Application No. 63/429,545, titled “X-RAY-BASED DENTAL CASE ASSESSMENT,” filed on Dec. 1, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to dental case assessment, and more specifically to x-ray based dental case assessment.

### BACKGROUND

Dental treatments, including orthodontic treatments, using a series of patient-removable appliances (e.g., “aligners”) are very useful for treating patients. Treatment planning is typically performed in conjunction with the dental professional (e.g., dentist, orthodontist, dental technician, etc.), by generating a model of the patient's teeth in a final configuration and then breaking the treatment plan into a number of intermediate stages (steps) corresponding to individual appliances that are worn sequentially. This process may be interactive, adjusting the staging and in some cases the final target position, based on constraints on the movement of the teeth and the dental professional's preferences. Once the final treatment plan is finalized, the series of aligners may be manufactured corresponding to the treatment planning.

Determining whether a patient is a candidate for various dental (e.g., orthodontic) treatments can be a painstaking and time-consuming process that requires a clinician to at least study photographic images of the patient's dentition. In some cases, there may be no photographic detention images available. However, a patient may have accessible several x-ray images associated with their dental files. In some other cases, a dentist, orthodontist, dental practice, or the like may be desirous of assessing and recommending various dental treatments for a patient database. Individual assessment may be laborious, particularly when there are a substantial number of patients to assess.

Thus, there is a need for determining whether a patient or groups of patients are good candidates for various dental (e.g., orthodontic) treatments based on dental x-ray images.

### SUMMARY OF THE DISCLOSURE

Described herein are methods and apparatuses (e.g., systems, devices, etc., including software, hardware and/or firmware) that can assess a patient's x-ray image(s), and recommend one or more dental treatments based on these x-ray image(s). In some examples, these apparatuses and methods may include a machine learning agent (e.g., neural network) that is trained to predict a patient's dental characteristics such as tooth crowding, tooth spacing and the like based on the patient's x-ray image. As used herein, dental treatments include orthodontic treatments.

Traditional thinking holds that the severity of an orthodontic cases cannot be based on a flat x-rays alone, since typical (and in particular, panoramic) x-rays show primarily tooth density and it is difficult or impossible to fully understand the three-dimensional arrangement of teeth, and to understand intercuspation of teeth, which is important in understanding orthodontic relationships between teeth. These technical problems have prevented the reliable automation of the analysis of orthodontic severity using x-rays. Other technical challenges include determining the outer surfaces of teeth from x-ray images, as well as understanding the relationship between soft tissues and teeth. The methods and apparatuses (e.g., systems) described herein may address these technical difficulties and provide procedures and technical steps that, somewhat surprisingly, reliably permit the identification of orthodontic issues severe enough to require treatment from x-ray images (and in some cases, just one or more x-ray images). The methods and apparatuses described herein may successfully assess orthodontic severity and provide meaningful recommendations in a way that has not previously been possible. Being able to assess orthodontic severity from flat x-rays (e.g., panoramic x-rays) allows much more rapid and efficient screening for potential orthodontic cases based on information that is already available (e.g., x-rays). The screening can either be performed as a batch run over old cases or connected as part of an imaging (e.g., x-ray) system to permit screening of an individual in the dental office without the need to invest additional time in using photo-based case assessment or an intraoral scan.

Any of these methods and apparatuses may include preprocessing of the x-ray images may permit the otherwise “flat” x-ray image (2D images) to be analyzed by the trained network in order to determine orthodontic recommendations that otherwise require three-dimensional information, such as outer tooth shape, relative tooth arrangement and/or intercuspation (e.g., bite engagement between teeth on opposite jaws). Preprocessing may include segmentation of the teeth and other structures (including soft tissue structures, e.g., gingiva) from the x-ray image(s). In any of these methods and apparatuses preprocessing may include identifying soft and bony (e.g., teeth) structures. In some cases the method and/or apparatus may adjust the contrast to assist in identifying soft (or putative) soft tissue, teeth and/or bone.

In general, described herein are methods of assessing a patient's x-ray image. Any of these methods may include receiving one or more dental x-ray images, determining one or more dental characteristics based on the one or more x-ray images using a trained neural network, wherein the trained neural network is trained using a plurality of training x-ray images and corresponding dental attribute data, comparing the one or more dental characteristics to one or more treatment thresholds, and outputting a recommendation for at least one dental treatment based on the comparison of the one or more dental characteristics to the one or more treatment thresholds. The training x-ray images in any of these methods and apparatuses may be panoramic x-ray images.

In general, the dental characteristics may describe any characteristic that may be predicted by the trained neural network. The dental characteristics may be associated with a patient's dentition. For example, a dental characteristic may include or describe tooth crowding and/or tooth spacing. The tooth crowding and/or tooth spacing may be described with measurement value. Any of the measurements described herein may be described with any feasible system of units including millimeters and/or inches.

In any of the methods described herein, the dental characteristics may include or describe an Angle's classification of malocclusion. Angle's classification refers to the maximal intercuspal position (MIP) relationship of the teeth, and typically does not consider the condylar position. Angle's classification is a static relationship between the occluding surfaces of the teeth, and may be determined by the hand articulation of maxillary and mandibular casts in MIP.

In some other examples, the dental characteristics may include or describe a deep bite, an open bite, and/or a presence of root collisions. The dental characteristics may also describe an estimated bone density.

In general, the trained neural network may be trained with training data. The training data may include one or more x-ray images as well as dental attribute data that is associated with each of the x-ray images. The dental attribute data may describe various attributes, measurements, characteristics, and the like of the patient's dentition. For example, the dental attribute data may include or describe tooth crowding, where the crowding is described in millimeters. Furthermore, the training data may be limited to include anterior teeth, thereby emphasizing the importance of the anterior teeth over other teeth.

In any of the methods described herein, the dental attribute data may include or describe tooth spacing (e.g., spacing between at least two teeth as expressed in millimeters). In some examples, the dental attribute data may include or describe a deep bite, an open bite, and/or root collisions. In some other examples, the dental attribute data may include or describe bone density information associated with each of the plurality of training x-ray images.

In general, the plurality of training x-ray images may be limited to upper anterior teeth, lower anterior teeth, or a combination thereof. Furthermore, the dental x-ray images that are received may include at least one panoramic x-ray. In some examples, the dental x-ray images that are received may include a bitewing and periapical x-ray images and determining the one or more dental characteristics may include applying the trained neural network on the bitewing and periapical x-ray images separately from each other. In some cases, the use of one or more panoramic images may be particularly helpful, as the trained neural network may infer relative positions of the teeth within the dental arch despite being from a ‘flat’ x-ray image.

In any of the methods described herein, any of the dental x-ray images may be received through an application programming interface (API). Furthermore, any of the recommendations provided by the trained neural network. May be output through the API.

In some examples, the one or more received dental x-rays may include a plurality of dental x-ray images from a plurality of patient. Thus, the recommendation may include recommendations for dental treatments for a plurality of patients.

In general, the treatment thresholds may be associated with a particular user or clinician. For example, one or more of the treatment thresholds may be based on preferences related to a user's preferred dental treatments.

In some examples, the received x-ray images may include a plurality of bitewing and periapical x-ray images, wherein determining the one or more dental characteristics are performed for all x-ray images simultaneously.

Any of the methods and apparatuses described herein may segmented the X-ray images. For example, any of these methods and apparatuses may include and/or use a second neural network to segment teeth from the radiograph in order to normalize the image. Segmented out (e.g., identified) teeth may be used to normalize the images for comparison and/or use by the first neural network. For example, segmented teeth may be used to determine which teeth correspond to a subset of the patient's teeth (e.g., the anterior teeth) once identified from the segmented images, these images may be cropped (e.g., automatically cropped).

In general, any structure within the oral cavity may be segmented. For example, in addition to teeth, spaces between teeth, and/or overlapping teeth may be segmented and identified. The segmented images may be used directly or indirectly for measuring or otherwise quantifying one or more features of the images. For example, using the size of a pixel in the radiograph, crowding and spacing may be estimated directly. By detecting the amount of space between the upper and lower posteriors vs the upper and lower anterior teeth, overbite may be measured/approximated. Likewise, the other clinical conditions may be directly measured once we have segmented and numbered the teeth in the radiograph.

In general, segmentation of the teeth and/or other structures within the oral cavity may be performed in any appropriate manner. In some examples, a second neural network (trained neural network, e.g., trained on segmented x-ray images) may be used to segment the images. For example, A second neural network may be used to identify teeth from a radiographic image, either a segmentation model (identify each tooth by a mask, i.e. a collection of pixels of a single tooth), or a tooth detection model (identify each tooth by a few key points, e.g. tooth root and tooth ridge points) may be used. The area of interest may then be cropped, based on either the tooth segmentation or detection results.

For example, in some of the methods and apparatuses described herein segmenting may include segmenting the one or more dental x-ray images to identify individual teeth, spaces between the teeth and/or overlapping teeth. In some examples segmenting comprises segmenting using a second trained neural network. For example, any of these methods and apparatuses may be configured to segment the one or more dental x-ray images to identify individual teeth and normalizing the one or more images to the identified teeth. In some examples normalizing comprises cropping the one or more images to exclude regions outside of the identified individual teeth.

Also described herein are apparatuses configured to perform any of these methods. For example, described herein is an apparatus for assessing a dental x-ray image that may include a communication interface, one or more processors, and a memory storing instructions that, when executed by the one or more processors, causes the one or more processors to receive one or more dental x-ray images, determine one or more dental characteristics based on the at least on patient's x-ray image using a trained neural network, wherein the trained neural network is trained using a plurality of training x-ray images and corresponding dental attribute data, compare dental characteristics based on the at least on patient's x-ray image using a trained neural network, wherein the trained neural network is trained using a plurality of training x-ray images and corresponding dental attribute data, compare the one or more dental characteristics to one or more treatment thresholds, and output a recommendation for at least one dental treatment based on the comparison of the one or more dental characteristics to the one of more treatment thresholds.

Also described herein is software for performing any of these methods. For example, described herein are non-transitory computer-readable storage medium storing instructions that, when executed by one or more processors of a device, cause the device to receive one or more dental x-ray images, determine one or more dental characteristics based on the at least on patient's x-ray image using a trained neural network, wherein the trained neural network is trained using a plurality of training x-ray images and corresponding dental attribute data, compare dental characteristics based on the at least on patient's x-ray image using a trained neural network, wherein the trained neural network is trained using a plurality of training x-ray images and corresponding dental attribute data, compare the one or more dental characteristics to one or more treatment thresholds, and output a recommendation for at least one dental treatment based on the comparison of the one or more dental characteristics to the one of more treatment thresholds.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIG. 1 schematically illustrates one example of a machine-learning x-ray image assessment apparatus 100.

FIG. 2 is a flowchart showing an example method for training a neural network to assess one or more patient x-ray images.

FIG. 3A shows an example panoramic x-ray image that may be included the x-ray training data of FIG. 1.

FIG. 3B shows an example bitewing x-ray that may be included in the x-ray training data of FIG. 1.

FIG. 4 is a flowchart showing one example of a method for determining whether a patient is a candidate for a dental treatment.

FIG. 5 shows a block diagram of a device that may be one example of the machine-learning x-ray image assessment apparatus of FIG. 1.

### DETAILED DESCRIPTION

Images, such as dental images, are widely used in the formation and monitoring of a dental treatment plan. For example, some dental images may be used to determine a starting point of a dental treatment plan, or in some cases determine whether a patient is a viable candidate for any of a number of different dental treatment plans.

Described herein are apparatus (e.g., systems and devices, including software) and methods for training and applying a machine learning agent (e.g., a neural network) to assess a patient's dental x-ray image and determine if the patient may be a candidate for a dental procedure. The patient's dental x-ray image may include one or more panoramic, a bitewing, periapical, or any other feasible x-ray. A processor or processing node can execute one or more neural networks that have been trained to determine dental assessment data from the patient's dental x-rays. In some examples, the dental assessment data may include one or more numeric values regarding or describing a patient's dentition. In other examples, the dental assessment data may include an indication of features and/or dental characteristics included in the patient's dentition.

Any of the dental assessment data determined by the neural networks may be compared to various treatment thresholds. In some examples, if dental assessment data exceeds a treatment threshold, then a dental procedure may be recommended for the patient. Some treatment thresholds may correspond to particular dental therapies or procedures that may be provided by a clinician.

In some examples, a “batch” of patient dental x-ray images corresponding to a number of patients may be processed by the neural network. In this manner, several hundreds or thousands of patients may be quickly and easily assessed for various dental treatments. In some examples, an application programming interface (API) may provide an input/output interface for a processor or processing node to receive patient x-ray images and provide recommendations for dental therapies. In some cases, the API may be web-based enabling execution of the neural network to be performed remotely at a data center, corporate office, in a compute cloud, or the like.

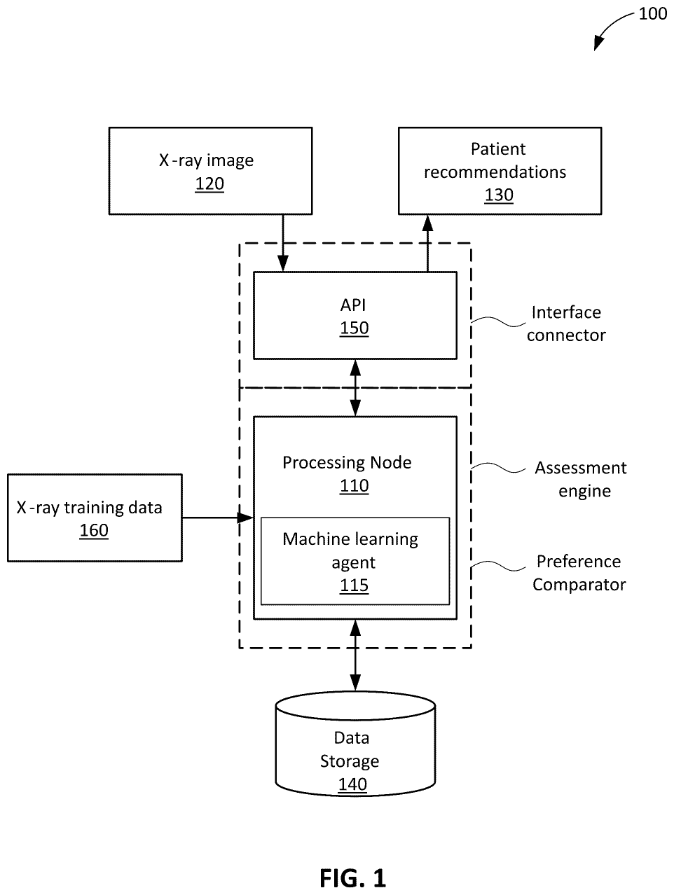

FIG. 1 schematically illustrates one example of a machine-learning x-ray image assessment apparatus 100. Although described herein as a system, the machine-learning x-ray image assessment apparatus 100 may be realized with any feasible apparatus, e.g., device, system, etc., including hardware, software, and/or firmware. In some examples, the machine-learning x-ray image assessment apparatus 100 may include a processing node 110, an application programming interface (API) 150, and a data storage module 140. As shown, the API 150 and the data storage module 140 may each be coupled to the processing node 110. In some examples, all components of the machine-learning x-ray image assessment apparatus 100 may be realized as a single device (e.g., within a single housing). In some other examples, components of the machine-learning x-ray image assessment apparatus 100 may be distributed within separate devices. For example, the coupling between any two or more devices, nodes (either of which may be referred to herein as modules), and/or data storage modules may be through a network, including the Internet. In this manner, the machine-learning x-ray image assessment apparatus 100 may be configured to operate as a cloud-based apparatus where some or all of the components of the machine-learning x-ray image assessment apparatus 100 may be coupled together through any feasible wired or wireless network, including the Internet.

The machine-learning x-ray image assessment apparatus 100 may include an interface connector to facilitate the receiving or input of patient x-ray images 120 and the outputting of patient recommendations. In some examples, the API 150 may provide the functionality of the interface connector. The machine-learning x-ray image assessment apparatus 100 may also include an assessment engine and a preference comparator. The assessment engine may assess the patient x-ray images 120 using one or more neural networks. The preference comparator may compare the outputs of the neural networks to one or more thresholds to determine if a patient is a candidate for a dental treatment. In some examples, the processing node 110 may provide the functionality of the assessment engine and the preference comparator.

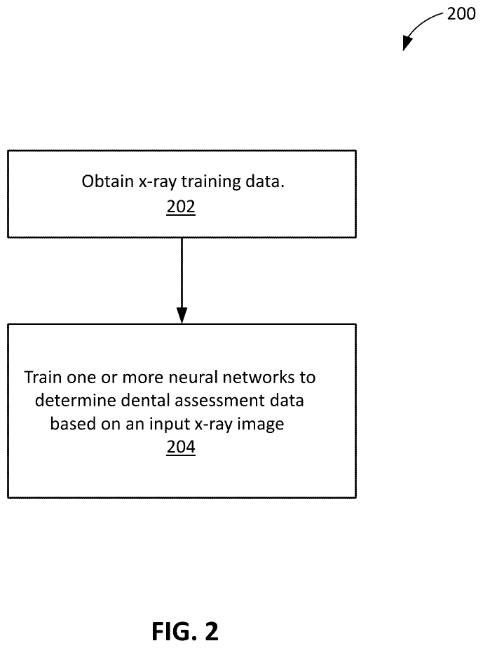

Thus, the API 150 can receive or obtain one or more patient x-ray images 120 and provide these images to the processing node 110. The processing node 110 may execute one or more neural networks to assess the patient x-ray images 120 for dental characteristics. The dental characteristics may include an indication of the presence of any one or more feasible dental characteristics. In some examples, the dental characteristics may also or alternatively include a measurement of any feature or features that may be associated with any dental, or oral anatomical characteristics. Dental characteristics are described in more detail below in conjunction with FIG. 2.

If a patient has certain dental characteristics (e.g., particular dental characteristics or measurements), then that patient may be a good candidate for one or more dental treatments. By way of a non-limiting example, if a patient has a predetermined amount (e.g., measurement or degree) of tooth crowding or tooth spacing, then that patient may respond well to orthodontic treatment with one or more aligners. Therefore, in some examples the processing node 110 can compare dental characteristics, provided by the trained neural networks, to various thresholds to determine whether a patient is a good candidate for various dental treatments.

In some examples, the various thresholds may be selected by the user. The user can select thresholds that correspond to ranges of dental characteristics for which the user feels comfortable or desirous of treating. For example, if a patient x-ray image 120 has a dental characteristics greater than (or in some cases less than) a threshold, then the processing node 110 may indicate that that patient is a candidate for a dental treatment.

Thus, the processing node 110 can output recommendations for dental treatments for patients with dental characteristics that are greater than, less than, or within thresholds (referred to herein as treatment thresholds). The processing node 110 can output these recommendations through the API 150.

The data storage module 140 may be any feasible data storage unit, device, structure, including random access memory, solid state memory, disk-based memory, non-volatile memory, and the like. The data storage module 140 may store image data, including patient x-ray images 120 data received through the API 150.

The data storage module 140 and/or the processing node 110 may also include a non-transitory computer-readable storage medium stores instructions that may be executed by the processing node 110. For example, the processing node 110 may include one or more processors (not shown) that execute instructions stored in the data storage module 140 to perform any number of operations including operations for assessing the patient x-ray images 120 and generating patient treatment recommendations 130. For example, the data storage module 140 may store one or more neural networks that may be trained and/or executed by the processing node 110. Alternatively, the processing node 110 may include one or more machine-learning agents 115 (e.g., trained neural networks, as descried herein), as shown in FIG. 1.

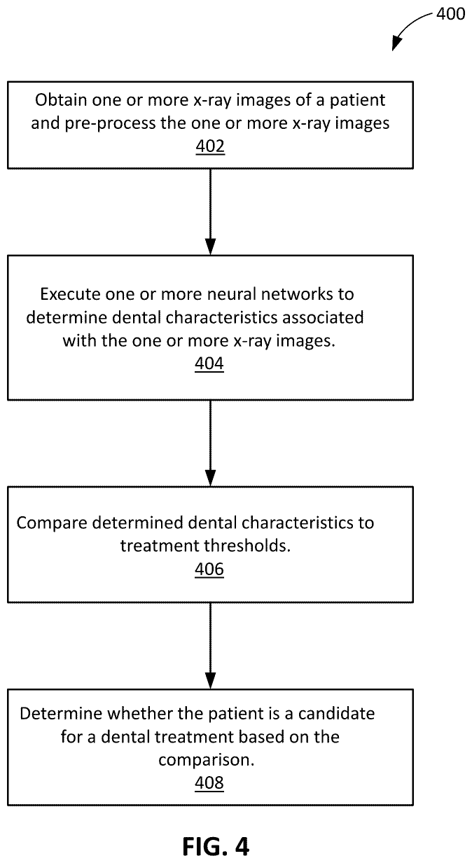

In some examples, the data storage module 140 may include instructions to train one or more neural networks to assess patient x-ray images 120. More detail regarding training of the neural networks are described below in conjunction with FIG. 2. Additionally, or alternatively, the data storage module 140 may include instructions to execute one or more neural networks to assess the patient x-ray images. More detail regarding the execution of a neural network is described below in conjunction with FIG. 4.

FIG. 2 is a flowchart showing an example method 200 for training a neural network to assess one or more patient x-ray images. Some examples may perform the operations described herein with additional operations, fewer operations, operations in a different order, operations in parallel, and some operations differently. The method 200 is described below with respect to the machine-learning x-ray image assessment apparatus 100 of FIG. 1, however, the method 200 may be performed by any other suitable apparatus, system, or device. In some examples, the neural network may be trained to assess or determine dental characteristics from a patient's x-ray image. The dental characteristics may include any feasible determined features (e.g., classifications) or dental numeric values.

The method 200 begins in block 202 as the processing node 110 obtains x-ray training data 160. The x-ray training data 160 may include dental x-rays (e.g., dental images) that show one or more aspects of a patient's dentition. For example, the x-ray training data 160 may include x-ray images of some or all of a person's teeth, soft tissue, bone structure, etc. The x-ray training data 160 may also include dental attribute data, such as dental numeric values and/or classification data associated with each x-ray image.

In some examples, the numeric values or classification data may include measured values or an indication regarding various dental characteristics. For example, the x-ray training data 160 may include a plurality of x-ray images and tooth crowding measurements that are associated with each x-ray image. All measurements described herein may be described in millimeters (mm), inches, or any other suitable measurement system. In another example, the x-ray training data 160 may include a plurality of x-ray images and tooth spacing measurements that are associated with each x-ray image. In yet another example, the x-ray training data 160 may include a plurality of x-ray images and Angle's classification of malocclusion (e.g., class 1, 2, or 3 descriptors of misalignment between the upper and lower dental arches) associated with each x-ray image.

In another example, the x-ray training data 160 may include a plurality of x-ray images and a measurement and/or classification/indication of an open bite associated with each x-ray image. In yet another example, the x-ray training data 160 may include a plurality of x-ray images and a measurement and/or classification/indication of a deep bite associated with each x-ray image. In another example, the x-ray training data 160 may include a plurality of x-ray images and a measurement and/or classification/indication of root collisions associated with each x-ray image.

In some examples, the x-ray training data 160 may include a plurality of x-ray images and a measurement of bone densities associated with each x-ray image. In some examples, the x-ray training data 160 may include a plurality of x-ray images and a determination of whether the images reflect a dentition that is amenable to one or more predetermined dental/orthodontic treatment.

Any of the x-ray training data 160 images described herein may include panoramic x-ray images, bitewing x-ray images, periapical x-ray images, or a combination thereof. In some examples, the x-ray training data 160 may be limited or cropped to substantially include the anterior teeth (e.g., teeth in the upper and/or lower dental arches between and including the canine teeth). In this manner, any neural network training may emphasize the priority or importance of the anterior teeth in the x-ray training data 160.

Next, in block 204 the processing node 110 may train one or more neural networks to determine (estimate or predict) dental characteristics based on an input x-ray image. In some examples, the dental characteristics may include numeric values and/or classification data associated with each x-ray image. In some cases, the neural networks may be trained to provide a regression value, a classification, or an ordinal classification based on an input x-ray image.

The processing node 110 can train a variety of neurons to recognize various aspects of the x-ray training data 160 and predict associated dental numeric values and/or dental classifications. In some examples, the processing node 110 may execute or perform any feasible supervised learning algorithm to train the neural network. For example, the processing node 110 may execute linear classifiers, support vector machines, decision trees or algorithms to predict dental classifications from an input x-ray image. The dental classifications may include, but are not limited to, Angle's classification, open bite, deep bite, and a determination of root collision.

In some other examples, the processing node 110 may execute or perform any feasible regression algorithm to train the neural network to predict tooth spacing, tooth crowding, bone density, or the like to determine any associated numeric values. In some examples, a loss function may be used to train a neural network to determine or predict possible dental numeric values.

In some examples, prior to training any neural network the processing node 110 may adjust a contrast or brightness associated with any images of the x-ray training data 160. Adjustment of the contrast or brightness may enable the processing node 110 to more easily detect any dental features (e.g., teeth, soft tissue including gingiva, etc.) in the x-ray training data 160. The trained neural networks may be stored in the data storage module 140.

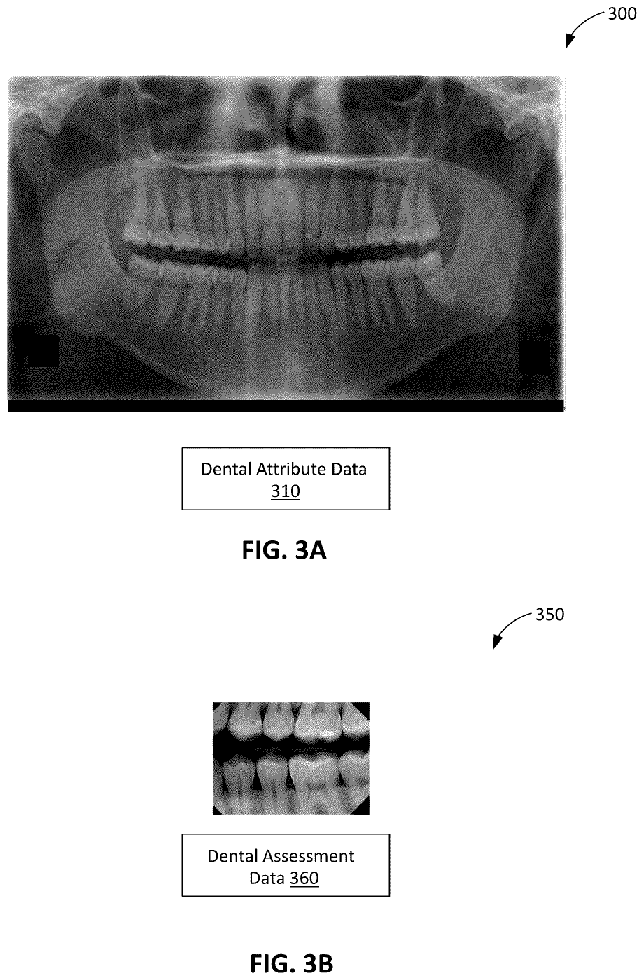

FIG. 3A shows an example panoramic x-ray image 300 that may be included the x-ray training data 160 of FIG. 1. The panoramic x-ray image 300 may include dental attribute data 310 (e.g., classification and/or numeric data) that is associated with the panoramic x-ray image 300. For example, the dental attribute data may include tooth spacing numbers, bone density values, or any other numeric values. The dental attribute data 310 may also or alternatively include classification data such as the presence of root collisions, open bite, deep bite and/or Angle's classification.

FIG. 3B shows an example bitewing x-ray 350 that may be included in the x-ray training data 160 of FIG. 1. Similar to as described for the panoramic x-ray image 300, the bitewing x-ray 350 may also include dental attribute data 360 associated with the bitewing x-ray 350.

FIG. 4 is a flowchart showing one example of a method 400 for determining whether a patient is a candidate for a dental treatment. The method 400 is described below with respect to the machine-learning x-ray image assessment apparatus 100 of FIG. 1, however, the method 200 may be performed by any other suitable apparatus, system, or device.

The method 400 begins in block 402 as the processing node 110 obtains one or more x-ray images of a patient. In some examples, the x-ray images may be a panoramic x-ray image of the patient. In some other examples, the x-ray images may include one or more bitewing or periapical x-rays of the patient. Any of these methods may include preprocessing of the one or more x-ray images of the patient. In some examples, the processing node 110 may adjust a contrast or brightness associated with any x-ray images of the patient. Adjustment of the contrast or brightness may enable the processing node 110 to more easily detect any dental features (e.g., teeth, soft tissue, including gingiva, etc.) in the x-ray images of the patient.

In general, pre-processing of the images may permit the “flat” x-ray image to be analyzed by the trained network in order to determine orthodontic recommendations that otherwise require three-dimensional information, such as outer tooth shape, relative tooth arrangement and/or intercuspation (e.g., bite engagement between teeth on opposite jaws). The methods and apparatuses described herein may address this technical problem by including one or more (or in some cases, the combination of) the use of panoramic x-ray image(s), pre-processing, including segmentation of the teeth from the x-ray image(s).

Next in block 404, the processing node 110 executes one or more neural networks to determine (e.g., estimate and/or predict) dental characteristics (numeric values or dental classifications) associated with the x-ray images received in block 402. As described with respect to FIG. 2, one or more neural networks may be trained to determine numeric dental values such as tooth spacing, tooth crowding, bone densities and the like from a patient's x-ray image. In some other examples, the one or more neural networks may be trained to determine whether one or more features (e.g., dental classifications) such as an open bite, deep bite, an Angle's classification, or the like are indicated by a patient's x-ray image.

If the patient's x-ray images include several bitewing or periapical images, then in some examples the processing node 110 may execute one or more neural networks separately on each image. Results of the individual neural network outputs may then be combined together. In another example, the processing node 110 may combine multiple x-ray images together to form a larger, more complete x-ray prior to execution of a neural network.

Next, in block 406 the processing node 110 compares the determined (estimated or predicted) dental characteristics to one or more treatment thresholds. The treatment thresholds may be associated with treatment preferences of a clinician, dental group, health organization, or the like. For example, a treatment threshold associated with tooth crowding may be greater than or equal to 2 mm. That is, patients having any tooth crowding more than 2 mm may be a candidate for a dental treatment. Other example numeric treatment thresholds may include thresholds for tooth spacing, bone density, and the like. Furthermore, treatment thresholds for dental classifications may include the presence or absence of root collisions, open bite, deep bite, or a particular Angle's classification.

In some examples, the treatment thresholds may be an ordinal classification. An ordinal classification may be related to a numeric dental value as ordered through a series of ordered classes. For example, a first class of tooth crowding may be tooth crowding values of less than 0.5 mm, a second class of tooth crowding may be tooth crowding values less than 1.0 mm, and a third class of tooth crowding may be any tooth crowding values less than 4.0 mm. In some examples, any one ordinal class may include the implication that lesser classes are true when a higher class is indicated. For example, if a second class of tooth crowding is determined or indicated, then the first class of tooth crowding is also indicated or determined.

Next, in block 408, the processing node 110 determines whether the patient is a candidate for a dental treatment based on the comparison of block 406. For example, if one or more numeric dental values or dental classifications (dental characteristics) exceed a treatment threshold, then the processing node 110 may determine that the patient is a candidate for a dental treatment. Because the treatment thresholds described in block 406 may be associated with the treatment preferences of a clinician, the processing node 110 may limit recommendations to treating dental cases within the preferences of the clinician.

For example, a clinician may want to treat patients for tooth crowding when the patient's tooth crowding measures at least 1 mm. The clinician may have determined that patients show little concern for procedures to correct tooth crowding when tooth crowding is less than 1 mm. Similarly, the clinician may want to treat patients for tooth crowding when the patient's tooth crowding measures less than 6 mm. The clinician may have determined that cases with tooth crowding greater than 6 mm are too complex to treat. Thus, cases of tooth crowding between 1 mm and 6 mm may be within a treatment preference region of a clinician. Therefore processing node 110 may be configured to recommend a dental treatment for tooth crowding cases between 1 mm and 6 mm.

Although a tooth crowding example is described here, the processing node 110 can recommend one or more dental treatments based on the values of various numerical values or dental classifications.

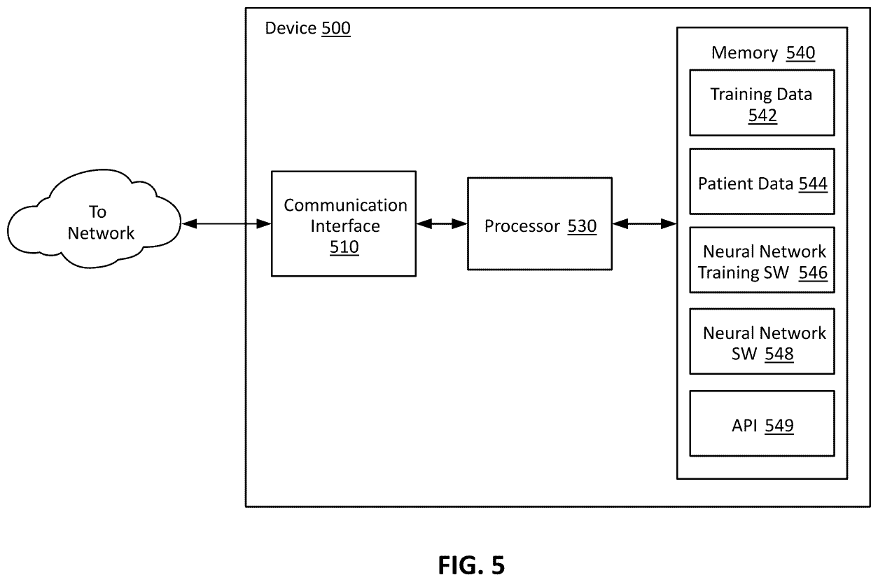

FIG. 5 shows a block diagram of a device 500 that may be one example of the machine-learning x-ray image assessment apparatus 100 of FIG. 1. Although described herein as a device, the functionality of the device 500 may be performed by any feasible apparatus, system, or method. The device 500 may include a communication interface 510, a processor 530, and a memory 540.

The communication interface 510, which may be coupled to a network and to the processor 530, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 510 may include wired (e.g., serial, ethernet, or the like) and/or wireless (Bluetooth, Wi-Fi, cellular, or the like) transceivers that may communicate with any other feasible device through any feasible network. In some examples, the communication interface 510 may receive training data 542 and/or patient data 544.

The processor 530, which is also coupled to the memory 540, may be any one or more suitable processors capable of executing scripts or instructions of one or more software programs stored in the device 500 (such as within memory 540).

The memory 540 may include training data 542. The training data 542 may include a plurality of x-ray images that include associated dental attribute data. As described above with respect to FIG. 2, the dental attribute data may include any feasible features (e.g., classifications) or numeric values. By way of example and not limitation, the dental attribute data may include tooth spacing values, tooth crowding values, Angle's classification values, the presence of open bite and/or deep bite, and/or root collisions.

The memory 540 may also include patient data 544. The patient data 544 may include one or more patient x-rays that are to be evaluated by the device 500 to determine if a dental treatment should be recommended for the associated patient. The patient data 544 may include panoramic x-rays, bitewing x-rays, periapical x-rays, or any other feasible x-rays. The patient data 544 may include the x-rays associated with a single patient, and/or may include the x-rays from several patients. In some examples, the device 500 may assess the x-rays from large groups of patients by processing the associated patient data 544 in a “batch mode.”

The memory 540 may also include a non-transitory computer-readable storage medium (e.g., one or more nonvolatile memory elements, such as EPROM, EEPROM, Flash memory, a hard drive, etc.) that may store a neural network training software (SW) module 546, a neural network SW module 548, and an API 549.

The processor 530 may execute the neural network training SW module 546 to train one or more neural networks to perform one or more of the operations discussed with respect to FIG. 2. In some examples, execution of the neural network training SW module 546 may cause the processor 530 to collect or obtain training data (such as x-ray images and associated dental assessment data within the training data 542) and train a neural network using the training data 542. The trained neural network may be stored as one or more neural networks in the neural network SW module 548.

The processor 530 may execute one or more neural networks in the neural network SW module 548 to assess patient x-ray images (which may be stored in the patient data 544) determine whether one or more dental treatments can be recommended for the patient. For example, execution of a neural network may assess a patient x-ray and determine dental characteristics (a numeric value and/or a dental characteristic/classification) associated with the patient's x-ray. Execution of the neural network may also include comparing the dental characteristics to treatment thresholds and generate treatment recommendations based on the comparison. In some examples, execution of a neural network in the neural network SW module 548 may cause the processor 530 to perform one or more of the operations discussed with respect to FIG. 4.

The processor 530 may execute instructions in the API 549 to receive patient x-ray images that may be included with the patient data 544 and output treatment recommendations that may be determined by executing the neural network SW module 548. In some examples, the API 549 may provide a web-based interface to receive and transmit x-ray images and recommendations.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various examples have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for assessing whether a patient is a candidate for a dental treatment, the method comprising:
receiving one or more panoramic dental x-ray images;
preprocessing the one or more panoramic dental x-ray images;
determining one or more dental characteristics based on the preprocessed one or more panoramic dental x-ray images using a trained neural network, wherein the trained neural network is trained using a plurality of training x-ray images and corresponding dental attribute data;
comparing the one or more dental characteristics to one or more treatment thresholds; and
outputting a recommendation for at least one dental treatment based on the comparison of the one or more dental characteristics to the one or more treatment thresholds.

2. The method of claim 1, wherein the one or more dental characteristics describes one or more of: a degree of tooth crowding described in millimeters, a degree of tooth spacing described in millimeters, an Angle's classification of malocclusion, a deep bite, an open bite, a presence of root collisions, and an estimated bone density.

3. The method of claim 1, wherein the corresponding dental attribute data on which the trained neural network is trained includes one or more of: tooth crowding in millimeters, tooth spacing in millimeters, Angle's classification of malocclusion, a deep bite, an open bite, root collisions, and bone density.

4. The method of claim 3, wherein the plurality of training x-ray images is limited to include images of anterior teeth.

5. The method of claim 1, wherein the plurality of training x-ray images is limited to upper anterior teeth, lower anterior teeth, or a combination thereof.

6. The method of claim 1, wherein the one or more panoramic dental x-ray images include a plurality of bitewing and periapical x-ray images and determining the one or more dental characteristics include applying the trained neural network on the bitewing and periapical x-ray images separately from each other.

7. The method of claim 1, wherein the one or more panoramic dental x-ray images are received through an application programming interface.

8. The method of claim 1, wherein the recommendation is output through an application programming interface.

9. The method of claim 1, wherein the one or more panoramic dental x-ray images include a plurality of x-ray images from a plurality of patients and the recommendation includes a recommendation for each of the plurality of patients.

10. The method of claim 1, wherein the one or more treatment thresholds are based on a user's preferences associated with the user's preferred dental treatments.

11. The method of claim 1, wherein comparing the one or more dental characteristics comprises adjusting the one or more treatment thresholds based a dental practitioner associated with the patient.

12. The method of claim 1, wherein the one or more panoramic dental x-ray images include a plurality of bitewing and periapical x-ray images, wherein determining the one or more dental characteristics are performed for all x-ray images simultaneously.

13. The method of claim 1, wherein preprocessing comprises segmenting the one or more panoramic dental x-ray images to identify individual teeth, spaces between the teeth and/or overlapping teeth.

14. The method of claim 13, where segmenting comprises segmenting using a second trained neural network.

15. The method of claim 1, wherein preprocessing comprises segmenting the one or more panoramic dental x-ray images to identify individual teeth and normalizing the one or more panoramic dental x-ray images to the identified individual teeth.

16. The method of claim 15, where normalizing comprises cropping the one or more panoramic dental x-ray images to exclude regions outside of the identified individual teeth.

17. A method for assessing whether a one or more patients of a group of patients are a candidate for a dental treatment, the method comprising:
receiving a batch of dental x-ray images corresponding to a group of patients, wherein for each patient there comprises one or more x-ray images including one or more panoramic x-ray images;
determining, for each patient of the group of patients, one or more dental characteristics based on the one or more x-ray images corresponding to each patient, using a trained neural network, wherein the trained neural network is trained using a plurality of training x-ray images and corresponding dental attribute data;
comparing, for each patient of the group of patients, the one or more dental characteristics to one or more treatment thresholds; and
outputting a dataset comprising recommendations for at least one dental treatment for one or more of the patients of the group of patients based on the comparison of the one or more dental characteristics for each patient of the group of patients to the one or more treatment thresholds.

18. An apparatus for assessing a dental x-ray image, the apparatus comprising:
a communication interface;
one or more processors; and
a memory coupled to the one or more processors, the memory storing computer-program instructions, that, when executed by the one or more processors, cause the one or more processors to:
receive one or more panoramic dental x-ray images;
pre-process the one or more panoramic dental x-ray images;
determine one or more dental characteristics based on the one or more panoramic dental x-ray images using a trained neural network, wherein the trained neural network is trained using a plurality of training x-ray images and corresponding dental attribute data;
compare the one or more dental characteristics to one or more treatment thresholds; and
output a recommendation for at least one dental treatment based on the comparison of the one or more dental characteristics to the one or more treatment thresholds.

19. A non-transitory computer-readable storage medium storing instructions that, when executed by one or more processors of a device, cause the device to:
receive one or more panoramic dental x-ray images;
preprocess the one or more panoramic dental x-ray images;
determine one or more dental characteristics based on the one or more panoramic dental x-ray images using a trained neural network, wherein the trained neural network is trained using a plurality of training x-ray images and corresponding dental attribute data;
compare the one or more dental characteristics to one or more treatment thresholds; and
output a recommendation for at least one dental treatment based on the comparison of the one or more dental characteristics to the one or more treatment thresholds.
